(19) United States
(12) Patent Application Publication
(10) Pub. No.: US 2026/0253703 A1
(43) Pub. Date: Aug. 27, 2026

(54) POSE TRAINING AND EVALUATION SYSTEM

(71) Applicant: POZE AI INC., New York, NY (US)

(72) Inventors: Gil Maor, New York, NY (US); Hadas Weisman, New York, NY (US)

(21) Appl. No.: 18/874,809

(22) PCT Filed: Jun. 16, 2023
(86) PCT No.: PCT/US2023/068566

§ 371 Date: Dec. 13, 2024

Related U.S. Application Data
(60) Provisional application No. 63/366,488, filed on Jun. 16, 2022.

(51) Int. Cl. G16H 20/30; G16H 30/20

(52) U.S. Cl. CPC G16H 20/30; G16H 30/20

(57) ABSTRACT

An augmented pose instruction system, system comprising; evaluation by an asynchronous analytical tool of a recorded video of a student's pose data; recommendation, via a recommendation engine, of corrections to poses of the student; approval of the corrections to the poses of the student, via an instructor dashboard; a visual report created of the corrections; and corrections communicated to the student account.

### CROSS-REFERENCE TO RELATED APPLICATIONS

This is a 371 application based on PCT/US2023/68566, filed Jun. 16, 2023 and claims priority to U.S. Provisional Application 63/366,488, filed on Jun. 16, 2022, all of which are incorporated herein by reference.

### BACKGROUND

Millions of users utilize desktop computers, laptop computers, smartphones, tablets, smart TV and other electronic devices on a daily basis to achieve personal fitness and rehabilitation goals. The consumption of online videos or streamed fitness and rehabilitation content is one-directional in nature and users have no effective means of getting real-time or offline feedback and analytics on how they performed the exercises, which poses they need to improve, and how to improve the execution of such poses. In addition, fitness and rehabilitation content providers and aggregators who publish such content have no effective means of interacting with the content's consumers to provide them with feedback on how they performed and to reengage them with offers of additional and tailored content or services. Pose training includes dance, yoga, martial arts, Pilates, calisthenics, weight training, Barre, and as well as a number of other fitness regimes. For example, as a primary problem area, we describe some of the pose challenges associated with one of these regimes; yoga and yoga instruction.

Yoga as exercise is a physical activity consisting mainly of poses/postures, often connected by flowing sequences, sometimes accompanied by breathing exercises, and frequently ending with relaxation lying down or meditation. Yoga in this form has become familiar across the world with a normative set of poses or asanas. An asana is a body posture, originally and still a general term for a sitting meditation pose, and later extended in hatha yoga and modern yoga as exercise, to any type of position, adding reclining, standing, inverted, twisting, and balancing poses.

### Yoga Sessions

Yoga sessions vary widely depending on the school and style, and according to how advanced the class is. As with any exercise class, sessions usually start slowly with gentle warm-up exercises, move on to more vigorous exercises, and slow down again towards the end.

A typical session in most styles lasts from an hour to an hour and a half, whereas in the Mysore style yoga, the class is scheduled in a three-hour time window during which the students practice on their own at their own speed, following individualized instruction by the teacher.

### At Home Instruction

Typical at home instruction is mostly a one directional event with a user of an online course watching a video or playing a prerecorded yoga session.

In creating the present solution the creators have recognized a number of problems. In the first instance the creators have identified that there is no easy way to address pose practice in a manner that provides real time feedback for the user with the quality level of an instructor's eye. The second problem that they identified is that the views of instructors may be relative or suggestive in a manner that there is variability in pose instruction between various instructors and there is also a lack of an absolute measure of pose that a user can reference in real-time while they are practicing by themselves.

Another problem arises in a recent development of online exercises that provides a two way video online class that offers everything from full classes to tutorials to meditation practices. However, in the case of two way video, the instructor view of the user is fixed and cannot engage with the user to adjust or correct pose style that is incorrect. Additionally if the instructor is supervising poses by more than a single client, the screen used may be very small and does not allow a full view of the client. In addition, where recordings of the users' exercises are available, the creators recognized that no solution provides a feasible or practical methodology for instructors to watch the full-length recordings and provide meaningful and tailored postural feedback to dozens, hundreds or potentially thousands of clients in a timely fashion.

### BRIEF SUMMARY

The present solution is an artificial intelligence driven teaching solutions solves the following problems for a user. The first problem solved is that a user can learn correct form, metre and new poses without an instructor present. As second problem solved is the user can either learn in an offline mode or a real-time teaching mode. A third problem solved is the ability to transition seamlessly from a human taught movement class to a practice session without an instructor without an additional setup for the user. A fourth problem solved is the instructors' ability to provide meaningful feedback in an asynchronous fashion based on the AI analysis of recordings of students' sessions and tailor such feedback for each student in a fraction of the time it would have taken to watch the full-length recording and provide individualized feedback to each student.

The proposed solution is a system and method of image processing, particularly utilizing machine learning and computer vision to provide feedback, instructions and analytics on the performance of fitness and rehabilitation exercises both in real-time and offline modes. The solution transforms the one-directional nature of online fitness and rehabilitation by providing users with an “AI companion” and fitness and rehabilitation content providers with an “AI extension”.

A user performs fitness or rehabilitation exercises captured by a recording device, the system recognizes the exercise the user is performing and then generates and provides visual, written or audio feedback on the exercises including instructions on how to correct specific poses. The system is content agnostic and can provide feedback and analytics on the performance of any fitness or rehabilitation exercises regardless of the method, sequence or instruction style; it is not depended on scripted sequences or on any pre-tagged poses and is capable of providing feedback and analytics on content that is created “on-the-fly” by the content provider or by the user.

The system enables fitness and rehabilitation content providers and aggregators to interact with users and provide them with tailored feedback and reengage them with additional content as well as a method to assess the quality of specific instructors and content. Users can share their progress and success with their social media communities including through sharing of recorded poses and achieve acknowledgement and/or rewards from fitness and rehabilitation content providers and aggregators for completing certain fitness or rehabilitation challenges.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an apparatus setup engaged with a user for a variant of the solution.

FIG. 2 illustrates pose calculation flow diagram of the solution in accordance with one embodiment.

FIG. 3 illustrates an aspect of real time pose calculation for the solution in accordance with one embodiment.

FIG. 4 illustrates a display variant of the solution in accordance with one embodiment.

FIG. 5 illustrates a user interface aspect of the solution in accordance with one embodiment.

FIG. 6 illustrates a user interface aspect of the solution in accordance with one embodiment.

FIG. 7 illustrates a user/instructor interface aspect of the solution in accordance with one embodiment.

FIG. 8 illustrates a user interface aspect of the solution in accordance with one embodiment.

FIG. 9 illustrates a user interface aspect of the solution in accordance with one embodiment.

FIG. 10 illustrates a machine learning aspect of the solution in accordance with one embodiment.

FIG. 11 illustrates a machine learning between environments aspect of the solution in accordance with one embodiment.

FIG. 12 illustrates a mobile device of the solution in accordance with one embodiment.

FIG. 13 illustrates a computer server aspect of the solution in accordance with a variant of the solution.

FIG. 14 illustrates a cloud computing aspect of the solution in accordance with one embodiment.

### DETAILED DESCRIPTION

### Methodology and Mode of Solution Operation

A number of Definitions are utilized throughout this specification with the following definitions:
Timestamp—a point in time from the beginning of the recorded or real-time fitness practice session.
Pose—a fitness or rehabilitation body pose (e.g., “plank”). Some poses have multiple variations.
Rep—the execution of a Pose (i.e., you could do a certain pose 10 times, each would be a rep). Each rep has a starting and ending timestamp.
Capture Device—any recording device, including a computer, laptop, webcam, smartphone, tablet, or other video recording device.
Content Delivery Device—any visual or audio device through which the user is consuming fitness or rehabilitation content based on which the user is performing poses, including computer, laptop, smartphone, tablet, Smart Television, AR/VR device or other visual and audio output device.
Content Enrichment Device—any device including wearable technologies providing additional information on the execution of fitness or rehabilitation exercises such as heart rate, breathing, etc.
Home—physical location of a user that is distant from the location of a fitness or rehabilitation provider.
Studio—physical location of a fitness or rehabilitation content provider.

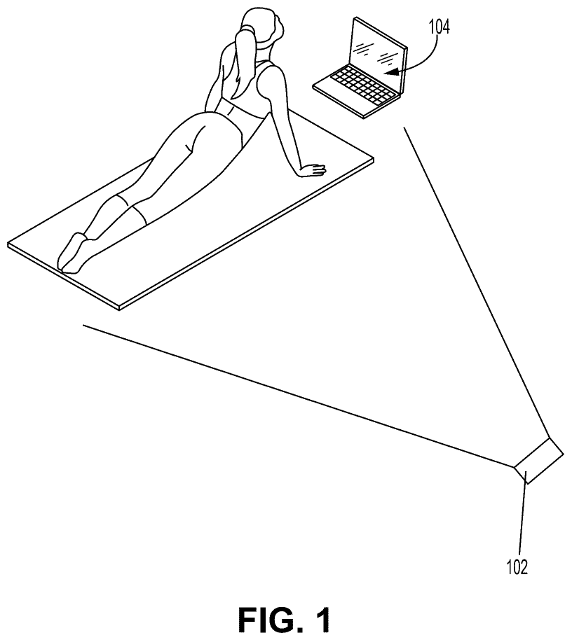

In FIG. 1, a user of the present solution is shown. A user in the privacy of their home that sets up a Content Delivery Device 104 and a Capture Device 102. The FIG. 1 shows the devices as a laptop and a mobile phone respectively as they are two devices readily available to many users. The creators also intend that Content Delivery Devices 104 could be chosen from a group that comprise desktop computers, smart televisions, mobile devices, netbooks, tablets, virtual, augmented, or extended reality devices, as well as other Content Delivery Devices that present the display of a set of poses for a user to follow.

Similarly, the Capture Device 102 is also selected from a group that comprises image and video capture aspects of desktop computers, smart televisions, mobile devices, netbooks, tablets, virtual, augmented, or extended reality devices, as well as other Capture Devices 102 that capture of a set of poses of a user for processing and display. The Capture Device 102 relays its information to a machine learning instance that performs algorithmic analysis and relays pose correction information via the Content Delivery Device 104 back to a user in one variant of the present solution although the Capture Device 102 (typically the smart phone with the camera) does not have to connect with or relay information to another Content Delivery Device 104 (e.g. the laptop). In practice, the Capture Device (e.g. smart phone) can also serve as a Content Delivery Device if the user chooses not to look at anything in front of her and is listens exclusively to the lesson that is broadcasted from the smartphone (i.e. the laptop is optional). In the real-time mode, the system offers the user a choice of audio feedback via the Capture Device 102 (e.g., the smartphone) for a response to the user's exercise poses. This feature provides auditory cues or instructions for pose corrections, focusing on accessibility and convenience should the user not wish to watch a screen. Should the user desire an in-depth understanding of their performance, they can optionally activate the visual feedback feature. This feature presents visual analytics and postural feedback in real time on the Content Delivery Device 104, enhancing the user's comprehension of their exercise form. The visual content includes the user's image captured by the Capture Device 102, overlaid by or presented near a posing avatar generated by machine learning analysis. Additionally, for offline analysis and postural feedback, users can log in to the cloud from any device. This integrated system, incorporating the Capture Device 102, and Content Delivery Device 104, linked between a cloud computing connection or an on premise server solution offers layered, customizable feedback options to suit the user's preferences and needs.

One can easily determine that this solution is useful in a virtual classroom setting as well where the pose correction is displayed to an instructor as an aid to help the instructor identify which student is struggling to master a pose. This could include a dashboard output to the Content Delivery Device 104 or alternatively Capture Device 102 (if so configured) providing functionality comprising the following metric features for users and instructors
1. Fitness and postural exercise providers have the option to customize the written and visual feedback that was generated by the system, via a generic or a white-labeled provider dashboard.
2. Fitness and postural exercise providers can pre-record their voice such that the auto-generated audio feedback is delivered in their own voice.
3. Fitness and postural exercise providers can offer asynchronous “private” sessions to multiple users, providing them with customized and tailored auto-generated audio, visual and written feedback.
4. Fitness and postural exercise providers can increase or decrease degree of sensitivity of the system to user limb positioning or to specific poses, thereby increasing or decreasing the level of feedback generated and to edit the system-generated feedback and augment it.
5. Based on the analytics generated by the system and the performance of the exercises by the users, fitness and exercise content aggregators and providers can create specific campaigns, promotions, rewards, and recommend users with additional content of interest.

### Other Use Cases for this Solution

1. End-user performing a fitness or postural exercise sequence at home desiring real-time feedback of pose corrections.
2. End-user performing a fitness or postural exercise sequence at home wishing to analyze their fitness or exercise session after-the-fact to find areas of improvement, snapshots of best achievements, or logging of fitness or exercise activities.
3. Fitness and exercise providers to reengage and interact with clients that perform exercise routines at home and provide them feedback to remove the one-directional nature of online training.
4. End-users performing individual or group fitness or exercise sequences in a studio or an exercise facility desiring real-time feedback of pose corrections; also allowing the provider to guide and supervise a larger number of users in real time.
5. End-user performing individual or group fitness or exercise sequences in a studio or an exercise facility wishing to analyze their fitness or exercise session after-the-fact to find areas of improvement, snapshots of best achievements, or logging of fitness or exercise activities.
6. Fitness and postural exercise providers to create fitness or exercise sequences and have them measured, analyzed and scripted out automatically.
7. Fitness and exercise content aggregators and providers to evaluate online fitness and exercise instructors by evaluating their sequences and their performance of the poses and generate best practices and create certification programs.
8. Fitness and exercise content aggregators and providers to create individual user and/or group challenges including rewardable challenges, using measurements such as rep scores and sequences.
9. ·End-user performing a fitness or postural exercise sequence at home using virtual, augmented, or extended reality devices for an immersive feedback and learning experience.
10. End-users wishing to share their fitness progress and achievements on social platforms for motivation and community engagement, using snapshots, video clips, or analytical data from their fitness or exercise sessions.
11. Fitness and exercise providers leveraging machine learning data generated by its clientele to customize and adapt exercise routines to individual users' abilities and progress, providing a personalized training experience.
12. Healthcare providers or physiotherapists utilizing the technology to monitor patients' rehabilitation exercises at home or at the clinic, providing rehabilitation corrections to ensure proper healing and recovery.
13. Schools and educational institutions incorporating the technology into physical education/dance instruction/gymnastics programs to provide students with feedback and to help instructors monitor students' performance.
14. ·Professional athletes or sports teams using the technology to analyze and improve their performance in specific sports movements or sequences.
15. Companies implementing the system as part of corporate wellness programs, enabling employees to receive real-time feedback on their exercises and track their progress.
16. Fitness and exercise content aggregators and providers utilizing the technology to offer gamified fitness experiences, where users can compete or cooperate in virtual challenges based on their actual exercise performance.
17. Fitness equipment manufacturers integrating the system with their products to provide users with real-time feedback and post-exercise analysis as part of the overall user experience.

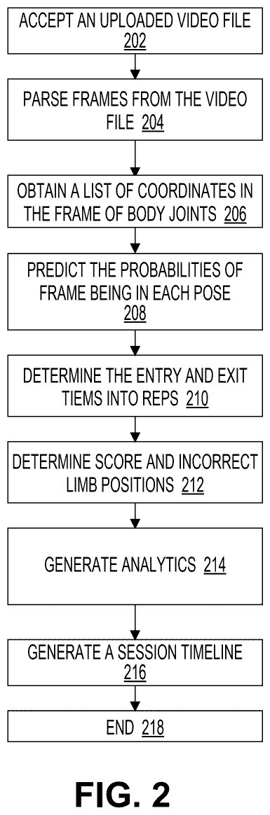

In FIG. 2 the present solution describes an Offline mode for a user which would provide episode level analysis at their option.

Offline mode: The user would typically set up their Content Delivery Device in front of a mat or clearing they use for their fitness or rehabilitation session, so that they face the device to see the content. The user would set up their Capture Device on the side of their mat or clearing, in order to capture a static side view of their practice session. Prior to starting a session, the user is prompted to review the automatically generated top tips to focus on based on their previous sessions.

The user would start a recorder app on their device, either a custom app or a generic video recording app. The user would then start their fitness or rehabilitation content on the Content Delivery Device, and proceed with their session while being recorded by the Capture Device from the side. After the session, the user would log into the system and upload the recorded video capturing their session.

The system would then accept an uploaded file 202 parse the video file frames 204 that was recorded using a Capture Device 102. Then using the individual frames from the video file, the solution obtains a list of coordinates in the frame of body joints 206. Then convert this list of coordinates into a list of features for a machine learning model that runs a pretrained Machine Learning model to predict the probabilities of frame being in each pose 208 Then go through the list of probabilities of being in a pose 208 at a given timestamp to determine the entry and exit time into reps 210. As certain poses have special handling as some are expected to be transitional (held for a brief period), restful (held for an extended period) or in a particular sequence (e.g., alternating each of the users legs), this solution allows for detected reps to be “padded” from both sides to calculate for demonstrating entry/exit in video clips. At this point, the solution, goes through list of reps to determine score and incorrect limb positions 212. Then the solution generates the following analytic calculations:
1. For each rep at each timestamp within the rep, compare each limb's angle to pretrained statistics of limb angles for that pose. Score rep at that timestamp based on the limbs' locations.
2. Pick the timestamp for an image representing this rep, based also on the time when best score was achieved.
3. Go through the limbs that were incorrectly positioned during this rep. Generate written feedback for the pose based on the limb positioning.
4. The system's degree of sensitivity to incorrect poses is set both automatically, based on the specifics of the pose and the general level of the user, and can be tailored by the user preference for variable sensitivity feedback.
5. Rank the best and worst poses.

Generate analytics 214 based on aggregate time in each pose and that pose's features (bend type, difficulty, muscles used). Generate a session timeline 216 based on the timestamps of the reps. Other variations of the Analytics Step 214 generates the following variations of the present solution.
Generate representative images and video clips of the reps based on scores, limb locations and pose feedback presented on Content Delivery Device 104.
Generate an avatar-like representation to display on the Content Delivery Device of the user performing the rep, highlighting which limbs were positioned correctly and those that are not correctly positioned and how to correct the pose.
A single device can be used simultaneously by the user as both the Content Delivery Device 104 and Capture Device 102 when the device is placed with a side view of the user.
The analytics and the resulting avatar-like representation on the Content Delivery Device 104 can be enriched with content received from integration with content enrichment further comprising graphical, textual or audio overlays to help the user enjoy their posing experience.

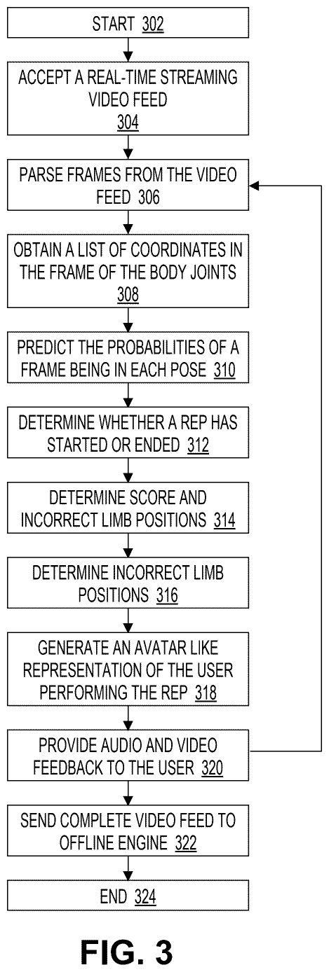

Real-time mode FIG. 3 describes an optional real-time mode of the present solution starting at 302. In this mode, the user would typically set up their Content Delivery Device 104 in front of a mat or clearing they use for their fitness or rehabilitation session, so that they face the device to see the content. The user would set up their Capture Device 102 on the side of their mat or clearing, in order to capture a static side view of their practice session. The user would start a custom app on the Capture Device, which would both record their session and provide the feedback.

Prior to starting a session, the user is prompted to review the automatically generated top tips to focus on based on their previous sessions.
The user would then start their fitness or rehabilitation content in the form of a real time streaming video feed 304 on the Content Delivery Device, and proceed with their session disregarding being recorded from the side.
The system would parse a real-time video feed 306 from a Capture Device 102, using a similar process as in offline mode. Obtaining a list of coordinates in the frame of the body joints 308
Determine pose entry and exit times in real-time, by predicting the probabilities of a frame being in each pose. 310 As it is difficult to calculate in real-time whether the ideal positioning in the rep was achieved, so we are looking at an arc of improvement and looking for when it had just past a peak pose. Additional steps include determining whether a rep has started or ended 312 Compare limb to ideal limb position. And generate a score 314 Find limbs that were incorrectly positioned 316, the solution generates an avatar figure showing an ideal pose versus the users pose, 318, then the solution provides audio feedback 320. Once audio feedback for the rep, the audio feedback set by the user to correct once or to continuously repeat the feedback until the limbs are positioned correctly.

After audio feedback for the rep has moved the user's limbs to a correct positioned, the solution then provides reinforcing feedback (e.g., “Good”). Then, if the user wishes to move to another pose, the solution then moves back to the parsing a real time video feed 306 step, to begin paring another pose and another set of limb detection. Once a session is complete, the solution sends the video recording to offline processing to achieve analytics as in offline mode. 322 A subsequent set of session feedback is provided in real time through a Content Delivery Device 104 Another variant of the solution is that a single device is used by the user as all device functions (Content Delivery and Content Capture) when the device is placed with a side view of the user. As this solution is not ideal for viewing, this variant user audio content and/or audio feedback rather than both audio and visual feedback.

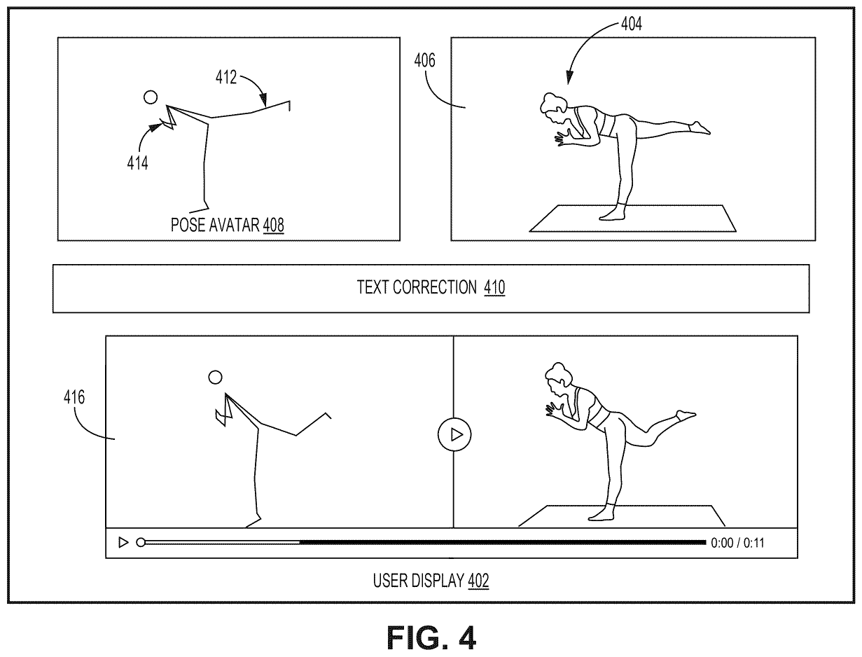

FIG. 4 is an exemplar of a home posing session User Display 402 presenting a Pose Avatar 408 stick figure representation of the pose captured by the Capture Device 102 of a User 404 in both image and video mode. The Captured Still Image 406 shows a User 404 striking a pose. The Pose Avatar 408 limb representations change color as the User 404 moves from an incorrect pose to an optimal pose position. In the present solution, Limb Segments 414 that are incorrectly positioned (e.g. Wrist to Elbow arm segment 412 and the right leg Knee-Ankle segment 408) would change color to red if in an incorrect position and then turn to “green” as the limb segment is moved to a corrected position. Additionally a Text correction 410 is written for a User 404 that wishes to read their corrections. The solution can accomplish this pose correction in a number of other ways using colors, strobes other methodologies to visually indicate to a user 404 to move into a better position. The Video Clip 416 shows a Pose Avatar 408 that is synchronized with the captured Video Clip 416.

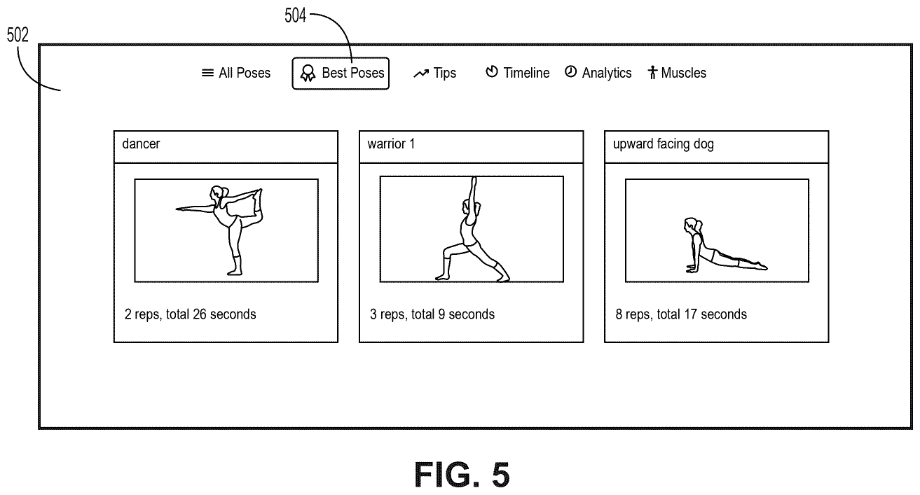

FIG. 5 shows an example user experience User Dashboard 502 by the solution showing a series of poses and metrics for each pose completed (e.g. Time, Reps, Pose Score) The User Dashboard 502 is shown a series of Best Poses 504 during a defined session period and allows a user to review items in their session or historical pose timeline, the associated analytics. The solution contemplates an episodic and continuum based version of this user interface where time intervals are developed to show a trend line of improvements.

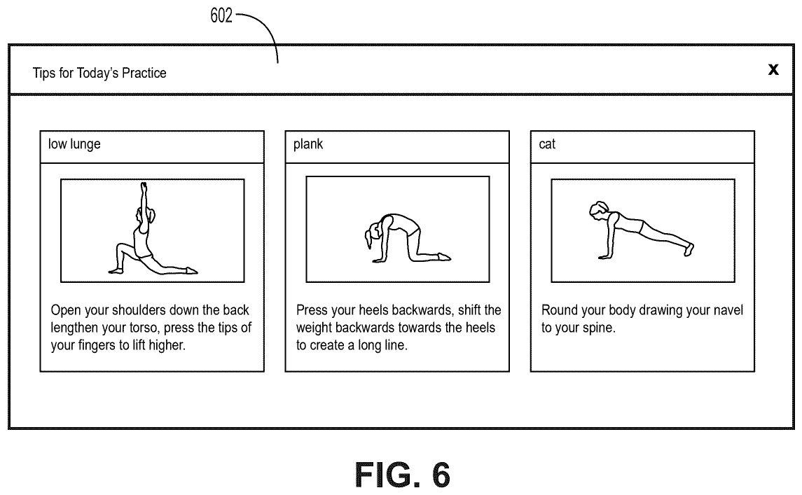

FIG. 6 shows a User Tips 602 viewable from a User Dashboard 502 summarizing the skills learned and corrected during a session. This user interface can either be instructor led or AI supervised to depending on the type of instruction system the present solution is integrated with.

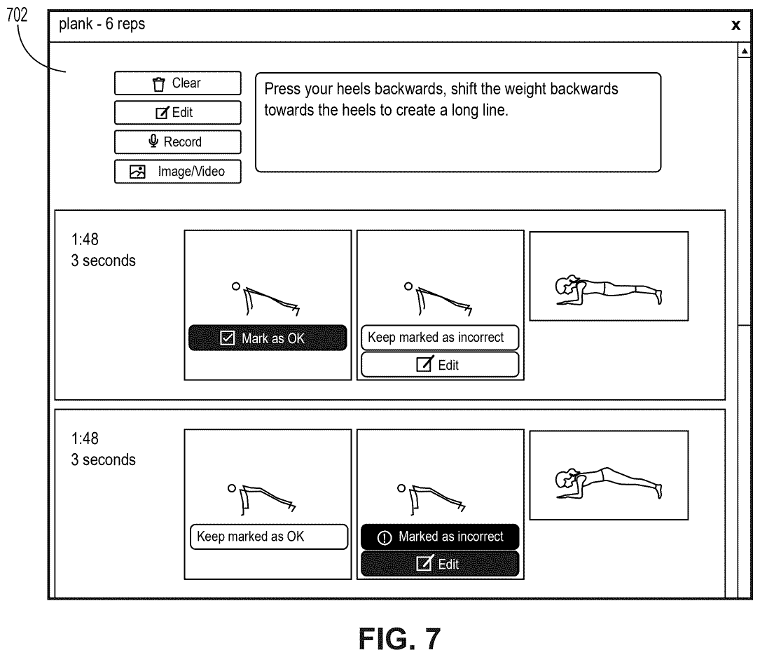

FIG. 7 is Instructional Display 702 describing a plank pose which is a strengthening and balancing pose that prepares the arms and core body for more advanced arm-balancing postures. In this interface the Pose Avatars 408 are shown in an evaluation format where the right most stick figures show the actual pose versus the desired pose for the student on the left most avatar.

This Instructional Display 702 is used by an instructor on how to perform the pose while also providing metrics and pose corrections as the user is either in the pose or reviewing the pose after completion of the session.

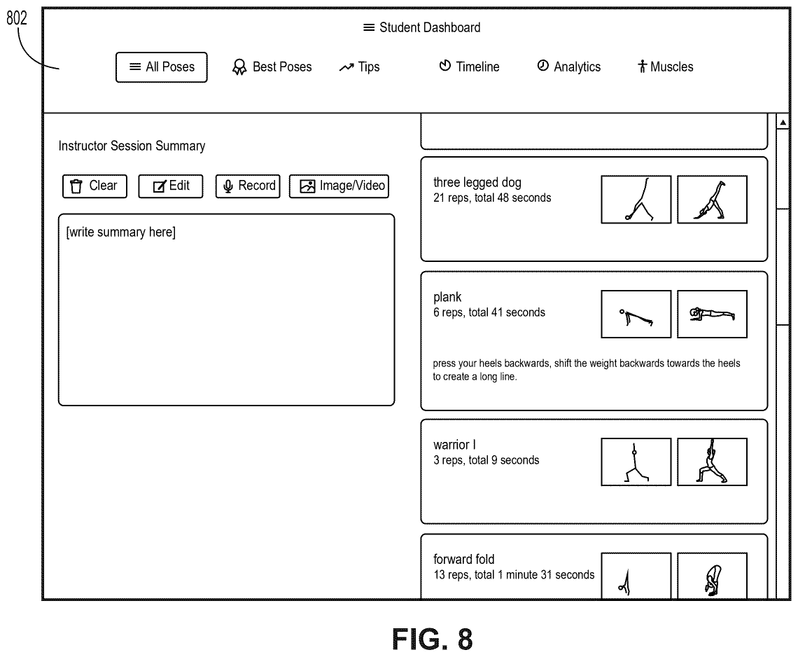

FIG. 8 is a user interface for a student showing a Pose Summary Display 802 to review and keep track of training sessions provided by the current solution showing various metrics, fitness summaries, timeline and other useful feedback for a user seeking to create a regime of exercise that uses pose mastery as a metric for the exercise. This display also allows an instructor offer qualitative commentary on top of the pose analytics.

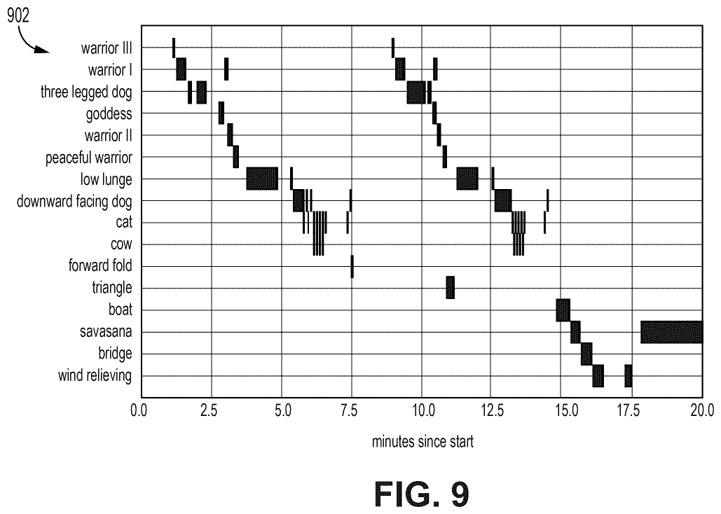

FIG. 9 is an example of the Composite Training Display 902 of the present solution. In this instance of the present solution, an exercise flow pattern of yoga poses is captured showing time, sequence, duration of poses and a color coding of pose mastery in time, quality and pose type as a minimum set of variables shown in composite format.

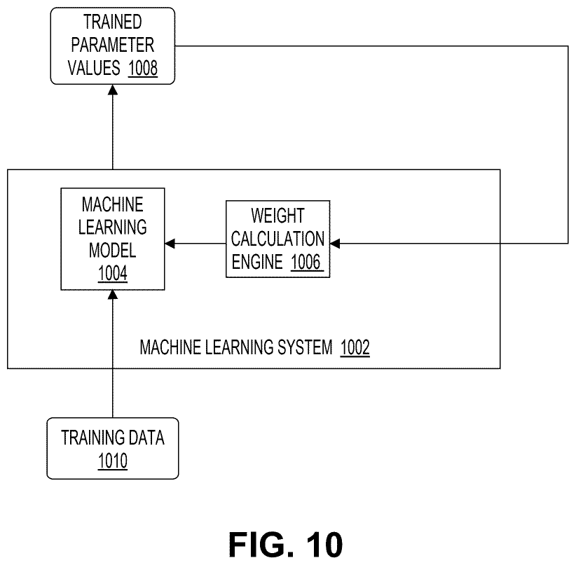

FIG. 10 shows an example Machine Learning System 1002. The Machine Learning System 1002 is an example of a system implemented as computer programs on one or more computers in one or more locations in which the systems, components, and techniques described below are implemented.

The Machine Learning System 1002 is configured to train a Machine Learning Model 1004 on multiple machine learning tasks sequentially. The Machine Learning Model 1004 can receive an input and generate an output, e.g., a predicted output, based on the received input.

In some cases, the Machine Learning Model 1004 is a parametric model having multiple parameters. In these cases, the Machine Learning Model 1004 generates the output based on the received input and on values of the parameters of the Machine Learning Model 1004.

In some other cases, the Machine Learning Model 1004 is a deep machine learning model that employs multiple layers of the model to generate an output for a received input. For example, a deep neural network is a deep machine learning model that includes an output layer and one or more hidden layers that each apply a non-linear transformation to a received input to generate an output. One variant of this solution uses a Convolution Neural Networks, Convolutional Neural Networks (CNNs) are a class of deep learning neural networks specifically designed for processing and analyzing visual data, such as images and videos. CNNs are widely used in computer vision tasks, including image classification, object detection, and image segmentation.

In general, the Machine Learning System 1002 trains the Machine Learning Model 1004 on a particular task, i.e., to learn the particular task, by adjusting the values of the parameters of the Machine Learning Model 1004 to optimize performance of the Machine Learning Model 1004 on the particular task, (e.g., by optimizing an objective function of the Machine Learning Model 1004.) The Machine Learning System 1002 can train the Machine Learning Model 1004 to learn a sequence of multiple machine learning tasks. Generally, to allow the Machine Learning Model 1004 to learn new tasks without forgetting previous tasks, the Machine Learning System 1002 trains the Machine Learning Model 1004 to optimize the performance of the Machine Learning Model 1004 on a new task while protecting the performance in previous tasks by constraining the parameters to stay in a region of acceptable performance (e.g., a region of low error) for previous tasks based on information about the previous tasks.

The Machine Learning System 1002 determines the information about previous tasks using a Weight Calculation Engine 1006 In particular, for each task that the Machine Learning Model 1004 was previously trained on, the Weight Calculation Engine 1006 determines a set of importance weights corresponding to that task. The set of importance weights for a given task generally includes a respective weight for each parameter of the Machine Learning Model 1004 that represents a measure of an importance of the parameter to the Machine Learning Model 1004 achieving acceptable performance on the task. The Machine Learning System 1002 then uses the sets of importance weights corresponding to previous tasks to train the Machine Learning Model 1004 on a new task such that the Machine Learning Model 1004 achieves an acceptable level of performance on the new task while maintaining an acceptable level of performance on the previous tasks.

Given that the Machine Learning Model 1004 has been trained on a first machine learning task, using first training data to determine first values of the parameters of the Machine Learning Model 1004, the Weight Calculation Engine 1006 determines a set of importance weights corresponding to task A. In particular, the Weight Calculation Engine 1006 determines, for each of the parameters of the Machine Learning Model 1004, a respective importance weight that represents a measure of an importance of the parameter to the Machine Learning Model 1004 achieving acceptable performance on task A. Determining a respective importance weight for each of the parameters includes determining, for each of the parameters, an approximation of a probability that a current value of the parameter is a correct value of the parameter given the first training data used to train the machine learning Machine Learning Model 1004 on task A.

For example, the Weight Calculation Engine 1006 determines a posterior distribution over possible values of the parameters of the Machine Learning Model 1004 after the Machine Learning Model 1004 has been trained on previous training data from previous machine learning task(s). For each of the parameters, the posterior distribution assigns a value to the current value of the parameter in which the value represents a probability that the current value is a correct value of the parameter.

In some implementations, the Weight Calculation Engine 1006 can calculate a posterior distribution using an approximation method, for example, using a Fisher Information Matrix (FIM). The Weight Calculation Engine 1006 can determine an FIM of the parameters of the Machine Learning Model 1004 with respect to task A in which, for each of the parameters, the respective importance weight of the parameter is a corresponding value on a diagonal of the FIM. That is, each value on the diagonal of the FIM corresponds to a different parameter of the Machine Learning Model 1004.

The Weight Calculation Engine 1006 can determine the FIM by computing the second derivative of the objective function at the values of parameters that optimize the objective function with respect to task A. The FIM can also be computed from first-order derivatives alone and is thus easy to calculate even for large machine learning models. The FIM is guaranteed to be positive semidefinite.

After the Weight Calculation Engine 1006 has determined the set of importance weights 120 corresponding to task A, the Machine Learning System 1002 can train the Machine Learning Model 1004 on new Training Data 1010 corresponding to a new machine learning task, e.g. task B.

To allow the Machine Learning Model 1004 to learn task B without forgetting task A, during the training of the Machine Learning Model 1004 on task B, the Machine Learning System 1002 uses the set of importance weights corresponding to task A to form a penalty term in the objective function that aims to maintain an acceptable performance of task A. That is, the Machine Learning Model 1004 is trained to determine Trained parameter Values 1008 that optimize the objective function with respect to task B and, because the objective function include the penalty term, the Machine Learning Model 1004 maintains acceptable performance on task A even after being trained on task B.

When there are more than two tasks in the sequence of machine learning tasks, e.g., when the Machine Learning Model 1004 still needs to be trained on a third task, e.g., task C, after being trained on task B, after the Trained parameter Values 1008 for task B have been determined, the Machine Learning System 1002 provides the Trained parameter Values 1008 to the Weight Calculation Engine 1006 so that the Weight Calculation Engine 1006 can determine a new set of importance weights corresponding to task B.

When training the Machine Learning Model 1004 on task C, the Machine Learning System 1002 can train the Machine Learning Model 1004 to use the set of importance weights corresponding to task A and the new set of importance weights corresponding to task B to form a new penalty term in the objective function to be optimized by the Machine Learning Model 1004 with respect to task C .. This training process can be repeated until the Machine Learning Model 1004 has learned all tasks in the sequence of machine learning tasks.

In the present solution, the training process is presented next
1. Images of fitness poses are labeled by the pose performed.
2. The images are run through a pose detection process to obtain a list of coordinates of the body joints in the image.
3. The coordinates of the body joints are transformed into features describing the location of the limbs.
4. A machine learning classifier is trained on the image features. This is used by the app to later recognize the user's pose.
5. For each pose measure the ideal angle of each limb. This is later used in the app to measure the correctness of the user's limb in the pose.

From an architectural stand point, this solution would be architected by creating a pose instructing apparatus that comprises a processing unit, a memory unit, and a plurality of modules that implement the functionality of the solution. An example would comprise the processing unit that executes instructions stored in the memory unit to facilitate the following functionalities:
Asynchronous analytical module: The asynchronous analytical module evaluates a recorded video of a student's pose. The module uses computer vision and machine learning techniques to identify the student's body parts and their positions. The module also identifies any errors in the student's pose. One methodology used is a trigonometric method to derive the relation between at least a joint and a limb is to use the following steps: 1) Identify the joint and the limb. The joint is the point where two bones meet, and the limb is the bone that is attached to the joint; b) Measure the angles between the bones. The angles can be measured using a protractor. Use trigonometry to calculate the length of the limb. The length of the limb can be calculated using the sine, cosine, or tangent mathematical relationships
Recommendation engine module: The recommendation engine module uses the information from the asynchronous analytical module to suggest corrections to the student's pose. The module uses a variety of factors to generate the suggestions, such as the student's level of experience, the type of pose being performed, and the specific errors that were identified.
Instructor dashboard module: The instructor dashboard module allows an instructor to approve or reject the suggested corrections. The instructor can also add their own comments to the suggestions.
Report generation module: The report generation module creates a report of the approved corrections. The report includes the student's name, the date of the evaluation, and the type of pose being performed, the errors that were identified, and the suggested corrections.
Communication module: The communication module sends the approved corrections to the student's account. The communication module can send the corrections via email, text message, or a mobile app.

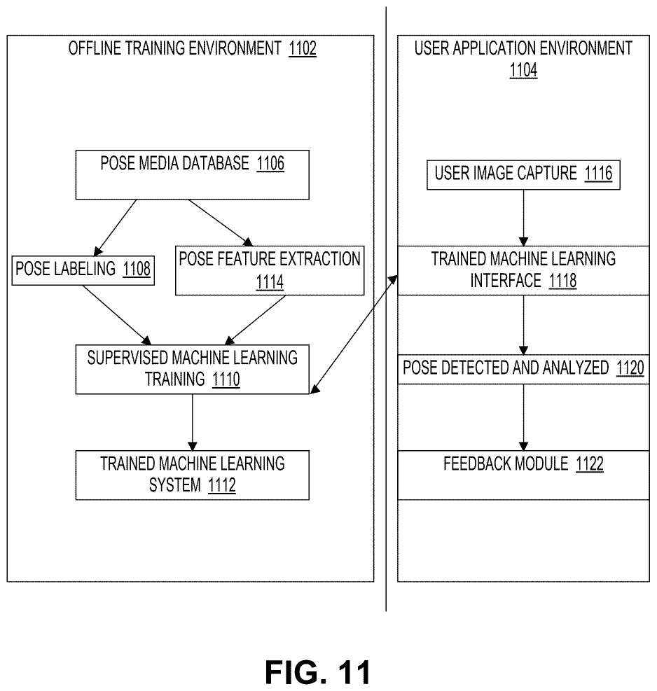

In FIG. 11 an process used explicitly by the present solution to train or evaluate poses in two environments 1) the Offline Training Environment 1102 and the User Application Environment 1104 The Offline Training Environment 1102 components comprise a Pose Media DataBase 1106 from which Pose Labeling 1108 and Pose Feature Extraction 1114 pull from to allow the Supervised Machine Learning Training 1110 driven by the Trained Machine Learning System 1112 to evaluate pose data(User Image Capture 1116) received from the User Application Environment 1104 via the Trained Machine Learning interface 1118 The Trained Machine Learning System 1112 compares received poses, generates avatars, sends the pose analysis back to a Pose detected/analyzed module 1120 that renders the avatar and instructs the Feedback module 1122 to communicate with and encourages the user with feedback and instructional support. A variant of this solution uses Support Vector Machines (SVMs) are supervised machine learning models that can be used for both classification and regression tasks. SVMs are particularly effective in handling high-dimensional data and finding a decision boundary that maximally separates different classes. Other variants may also use Gradient Boosting, which is a machine learning technique used for both regression and classification problems. It belongs to the ensemble learning methods and is based on the idea of sequentially combining weak learners, typically decision trees, to create a strong predictive model.

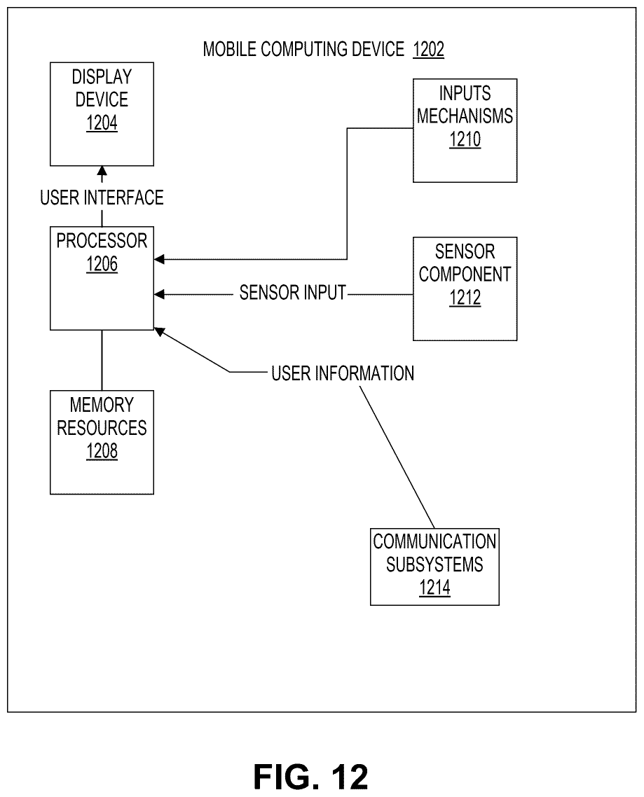

FIG. 12 is a block diagram that illustrates a mobile computing device upon which embodiments described herein may be implemented. In one embodiment, a Mobile Computing Device 1202 may correspond to a mobile computing device, such as a cellular device that is capable of telephony, messaging, and data services. Examples of such devices include smartphones, handsets or tablet devices for cellular carriers. Mobile Computing Device 1202 includes a Processor 1206, Memory Resources 1208, a Display Device 1204 (e.g., such as a touch-sensitive display device), one or more Communication SubSystems 1214 (including wireless communication sub-systems), input mechanisms (e.g., an input mechanism can include or be part of the touch-sensitive display device), and one or more Sensor Component 1212. In one example, at least one of the Communication SubSystems 1214 sends and receives cellular data over data channels and voice channels.

The Processor 1206 is configured with software and/or other logic to perform one or more processes, steps and other functions described with implementations, such as described by FIGS. Presented earlier, and elsewhere in the application. Processor 1206 is configured, with instructions and data stored in the Memory Resources 1208, to operate an on-demand service application as described in the other Figures For example, instructions for operating the service application to display various user interfaces, such as described in the earlier Figures, can be stored in the Memory Resources 1208 of the Mobile Computing Device 1202. In one implementation, a user can operate the on-demand service application so that sensor data can be received by the Sensor Component 1212. The sensor data can be used by the application to present user interface features that are made specific to the position and orientation of the Mobile Computing Device 1202 The sensor data can also be provided to the posing service system using the Communication SubSystems 1214. The Communication SubSystems 1214 can enable the Mobile Computing Device 1202 to communicate with other servers and computing devices, for example, over a network (e.g., wirelessly or using a wire). The sensor data can be communicated to the pose service system so that when the user requests the on-demand pose service, the system can arrange the service between the user and an available service provider. The Communication SubSystems 1214 can also receive user in formation (such as location and/or movement information of pose users in real-time) from the pose service system and transmit the user information to the Processor 1206 for display a user's data on one or more user interfaces.

The Processor 1206 can cause user interface features to be presented on the Display Device 1204 by executing instructions and/or applications that are stored in the Memory Resources 1208. In some examples, user interfaces, such as user interfaces described with respect to earlier FIGS can be provided by the Processor 1206 based on user input and/or selections received from the user. In some implementations, the user can interact with the touch-sensitive Display Device 1204 to make selections on the different user interface features so that pose-specific information (that is based on the user selections) can be provided with the user interface features. While FIG. 12 is illustrated for a mobile computing device, one or more embodiments may be implemented on other types of devices, including full-functional computers, such as laptops and desktops (e.g., PC).

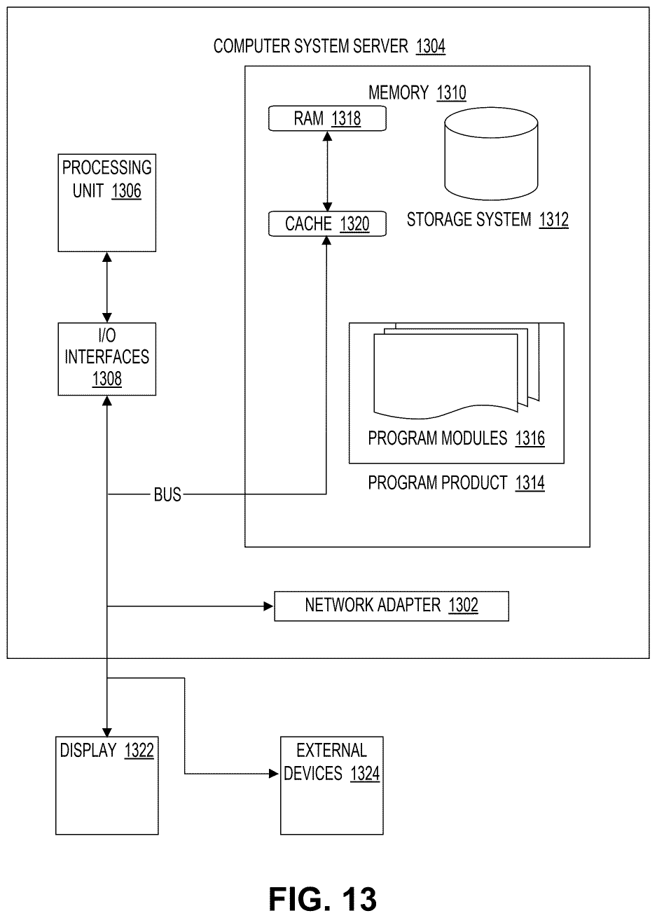

FIG. 13 is a general description of a server instance for implementing the present solution.

Computer System Server 1304 may be described in the general context of computer system-executable instructions, such as program modules, being executed by a computer system. Generally, program modules may include routines, programs, objects, components, logic, data structures, and so on that perform particular tasks or implement particular abstract data types. Computer System Server 1304 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 13, Computer System Server 1304 is shown in the form of a general-purpose computing device. The components of Computer System Server 1304 may include, but are not limited to, one or more processors or Processing Unit 1306 a Memory 1310 and a bus that couples various system components including Memory 13108 to Processing Unit 1306.

Bus represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer System Server 1304 typically includes a variety of computer system readable media. Such media may be any available media that is accessible by Computer System Server 1304 and it includes both volatile and non-volatile media, removable and non-removable media.

Memory 1310 can include computer system readable media in the form of volatile memory, such as random access memory (RAM 1318) and/or memory in the Cache 1320. Computer System Server 1304 may further include other removable/non-removable, volatile/non-volatile computer system storage media (e.g. Storage System 1312). By way of example, Storage System 1312 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus by one or more data media interfaces. As will be further depicted and described below, Memory 1310 may include at least one program product having a set (e.g., at least one) of program modules that are configured to carry out the functions of embodiments of the solution.

The solution Memory 1310 includes at least one Program product 1314 having a set (e.g., at least one) of Program Modules 1316 that are configured to carry out the functions of embodiments of the solution. They are stored in Memory 1310 by way of example, and not limitation, as well as an operating system, one or more application programs, other program modules, and program data. Each of the operating system, one or more application programs, other program modules, and program data or some combination thereof, may include an implementation of a networking environment. Program modules 1316 generally carry out the functions and/or methodologies of embodiments of the solution as described herein.

Computer System Server 1304 may also communicate with one or more External Devices 1324 such as a keyboard, a pointing device, a Displays 1322, etc. ; one or more devices that enable a user to interact with Computer System Server 1304; and/or any devices (e.g., network card, modem, etc.) that enable Computer System Servers 1304 to communicate with one or more other computing devices. Such communication can occur via Input/Output I/O interfaces 1308. Still yet, Computer System Server 1304 can communicate with one or more networks such as a local area network (LAN), a general wide area network (WAN), and/or a public network (e.g., the Internet) via Network a. As depicted, network adapter communicates with the other components of Computer System Server 1304 via bus. It should be understood that although not shown, other hardware and/or software components could be used in conjunction with Computer System Server 1304 Examples, include, but are not limited to: microcode, device drivers, redundant processing units, external disk drive arrays, RAID systems, tape drives, and data archival storage systems, etc.

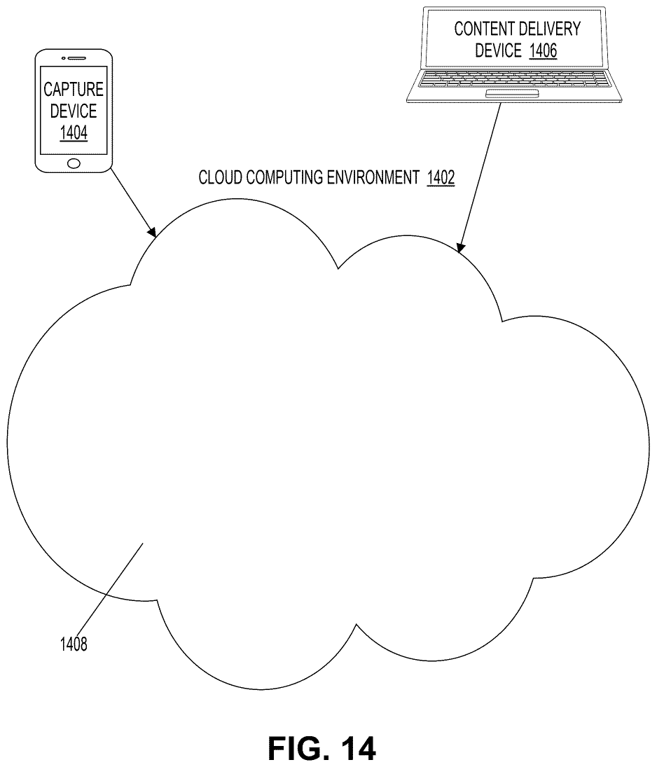

Referring now to FIG. 14, illustrative a Cloud Computing Environment 1402 is depicted. As shown, Cloud Computing Environment 1402 comprises one or more Cloud Computing Node 1408 with which local computing devices used by cloud consumers, such as, for example, a Capture Device 1404 or a Content Delivery Device 1406 can communicate. Cloud Computing Nodes 1408 may communicate with one another. They may be grouped (not shown) physically or virtually, in one or more networks, such as private, community, public, or hybrid clouds as described hereinabove, or a combination thereof.

This allows Cloud Computing Node 1408 to offer infrastructure, platforms, and/or software as services for which a cloud consumer does not need to maintain resources on a local computing device. It is understood that the types of computing devices are intended to be illustrative and that Cloud Computing Nodes 1408 and Cloud Computing Environment 1402 can communicate with any type of computerized device over any type of network and/or network addressable connection (e.g., using a web browser).

The primary mode of this solution is a solution that exists in a cloud instance. Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics of the present solutions cloud instance can include:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (Saas): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models for the present solution are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes The present solution may be a system, a method, and/or a computer program product at any possible technical detail level of integration. The computer program product may include a computer readable storage medium (or media) having computer readable program instructions thereon for causing a processor to carry out aspects of the present solution.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present solution may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present solution.

Aspects of the present solution are described herein with reference to flowchart illustrations and/or block diagrams of methods, apparatus (systems), and computer program products according to embodiments of the solution. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present solution. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

1. A computer-based method for calculating a pose of a human, the method comprising:
receiving a video stream of the human;
detecting at least a part of the human posing;
identifying a specific limb of the human;
applying a comparator to compare a position of the identified limb to a trained position;
issuing a corrective instruction for correction of the pose;
and tracking the user change in the position of the identified limb in response to the issued corrective instruction.

2. The computer-based method of claim 1 further comprising:
parsing the video stream into a set of frames;
detecting and identifying at least the part of the human posing within a frame or sequence of frames in the set of frames;
identifying the specific limbs of the human being and a specific joint using a deep learning methods that are pre-trained to recognize and locate a set of different body parts;
applying a trigonometric method to derive a relation between at least a joint and a limb; and
applying the comparator to compare a vector of the identified limb to train the vectors of the same limb using machine learning methods.

3. The computer-based method of claim 2 further comprising: identifying an incorrect limb movement for a given pose; calculating the correct method of achieving the given pose, based on angles and distances related to the limb and the joint; and using a set of known limb positions to identify whether the limb is in a correct position or otherwise requires correction.

4. The computer-based method of claim 3 wherein issuing an instruction for correction of the pose is generated using a cue further comprising of audio or visual prompts.

5. The computer-based method of claim 4 wherein tracking a change in the position of the identified limb in response to the issued corrective instruction is performed iteratively as the identified limb moves.

6. A pose instruction system, comprising:
an asynchronous analytical tool configured to evaluate a recorded video of a student's pose;
a recommendation engine configured to suggest corrections to the student's poses;
an instructor dashboard configured to approve the suggested corrections to the student's poses;
a report generator configured to create a report of the approved corrections;
and a communication module configured to send the approved corrections to a student's account.

7. The pose instruction system of claim 6 wherein the instructor dashboard further comprises a review interface configured to display information on a plurality of students, wherein the recommendation engine is configured to automate the suggestion of corrections.

8. The pose instruction system of claim 6 wherein the instructor dashboard is fully automated via the recommendation engine to emulate an instructor.

9. The pose instruction system of claim 6 wherein the instructor dashboard is designed to mimic a human instructor's instruction.

10. The pose instruction system of claim 6 wherein the system is configured to measure over time whether an exercise regime benchmark associated with the student is achieved.

11. The pose instruction system of claim 10, wherein the system is configured to measure whether a certification requirement was met for at least one student.

12. An apparatus for instructing poses, the apparatus comprising:
a processing unit;
a memory unit storing instructions that, when executed by the processing unit, facilitate a set of functionalities:
an asynchronous analytical module to evaluate a recorded video of a student's pose;
a recommendation engine module to suggest corrections to the student's poses;
an instructor dashboard module to approve the suggested corrections to the student's poses;
a report generation module to create a report of the approved corrections; and
a communication module to send the approved corrections to a student's account.

13. The apparatus of claim 12 wherein the instructor dashboard module further comprises a review interface module configured to display a set of pose information on a plurality of students, and wherein the recommendation engine module automates the suggestion of corrections.

14. The apparatus of claim 12 wherein the instructor dashboard module is further configured to receive an instructor's correction input to the recommendation engine module.

15. The apparatus of claim 14 wherein the recommendation engine module is configured to recommend at an least one training pose to the instructor.

16. The apparatus of claim 12 wherein the memory unit further stores instructions that, when executed by the processing unit, enable the apparatus to analyze a set of multiple poses of a plurality of students, and further comprising the recommendation engine module suggests an associated exercise regime for at least one student.

17. The apparatus of claim 12 wherein the memory unit stores instructions that, when executed by the processing unit, instructs the apparatus to measure over a time interval whether an exercise regime benchmark is achieved for one of the students, and whether a certification requirement was met for at least one of the students.

18. The apparatus of claim 12 wherein the instructor dashboard module is designed to mimic a human instructor's instruction.

19. The apparatus of claim 18 wherein the instructor dashboard module is fully automated via the recommendation engine module to emulate an instructor.

20. The pose instruction system of claim 10, wherein the instructor dashboard is designed to mimic a human instructor's instruction.
